(19) United States
(12) Patent Application Publication
(10) Pub. No.: US 2026/0137832 A1
(43) Pub. Date: May 21, 2026

(54) PREPARATION METHOD OF COLLAGEN MATRIX MEMBRANE AND APPLICATION THEREOF

(71) Applicant: Beijing Yiling Biotechnology Co., Ltd.

(72) Inventors: Lixin Xu, Beijing (CN); Hui Sun, Beijing (CN); Manqi Yin, Beijing (CN); Wenhao Zhang, Beijing (CN); Feixiang Cai, Beijing (CN)

(21) Appl. No.: 19/271,814

(22) Filed: Jul. 17, 2025

(30) Foreign Application Priority Data
Nov. 21, 2024 (CN) 202411667825.9

(51) Int. Cl. A61L 27/24; A61L 27/36; A61L 27/54

(52) U.S. Cl. CPC A61L 27/24; A61L 27/3629; A61L 27/54; A61L 2430/02

(57) ABSTRACT

Disclosed is a preparation method of a collagen matrix membrane and an application thereof. The invention uses animal intestinal casing as a raw material and sequentially includes steps such as decellularization and degreasing to prepare a collagen matrix membrane with excellent mechanical properties and a long degradation cycle. This membrane is more conducive to providing sufficient bone regeneration space and time during the initial healing phase of periodontal disease, better meeting clinical application needs. Additionally, the collagen matrix membrane prepared by the invention is loaded with multiple growth factors beneficial to periodontal tissue growth, thereby facilitating periodontal tissue regeneration and repair, and improving therapeutic efficacy.

### TECHNICAL FIELD

The invention relates to the technical field of biomembrane preparation, and particularly to a preparation method of a collagen matrix membrane and an application thereof.

### BACKGROUND ART

GTR membrane, namely Guided Tissue Regeneration Membrane, is a medical technical means used for periodontal tissue regeneration. GTR membrane regenerates periodontal structures by using a barrier membrane (periodontal regeneration membrane). Its core function is to isolate epithelium and connective tissue, providing time and space for osteoblasts to form new alveolar bone, cementum, and periodontal ligament. This technology is widely used in the clinical treatment of periodontal diseases, especially in treating gingival recession. The GTR membrane is a key factor for the successful implementation of GTR surgery and plays an irreplaceable role in the periodontal tissue regeneration process.

GTR membranes are classified as non-absorbable membranes/bioabsorbable membranes; they provide space for periodontal tissue regeneration. Non-absorbable membranes have gradually been phased out due to difficulty in degradation. Collagen membranes are common bioabsorbable membranes, but current collagen membranes have poor mechanical properties and degrade too rapidly in vivo, affecting the therapeutic efficacy of periodontal disease.

### SUMMARY OF THE INVENTION

Therefore, based on the above background, the invention provides a preparation method of a collagen matrix membrane and an application thereof. The membrane not only has excellent mechanical properties but also possesses a long degradation cycle, better meeting clinical application needs.

The technical solution of the invention is:
a preparation method of a collagen matrix membrane, including the following steps:
1) taking intestinal casing from a mammal, cleaning it, and soaking it in water;
2) soaking the intestinal casing from step 1) in a viral inactivation solution, followed by washing with purified water;
3) soaking the intestinal casing processed in step 2) in a degreasing solution multiple times, followed by washing with purified water;
4) soaking the intestinal casing processed in step 3) in a sodium dodecyl sulfate solution, followed by washing with purified water;
5) spreading the intestinal casing processed in step 4) flat, stacking multiple layers, pressing multiple times to form a membrane, and performing gradient freeze-drying with a freeze dryer.

Preferably, the mammal in step 1) is selected from one of pig, cow, or sheep.

Preferably, the viral inactivation solution in step 2) is selected from at least one of sodium hydroxide solution, sodium hypochlorite solution, formic acid, or hydrogen peroxide solution.

Preferably, the concentration of the sodium dodecyl sulfate solution in step 4) is 0.1-0.3 wt %.

When soaking in the sodium dodecyl sulfate solution in step 4), the material-to-liquid weight ratio of intestinal casing to sodium dodecyl sulfate solution is 1:(5-6), the soaking temperature is constant at 20-26° C., and the soaking duration is 3-9 hours.

Preferably, the degreasing solution in step 3) is prepared from chloroform and methanol in a volume ratio of (2-4):1.

Step 3) adopts soaking in a degreasing solution 2-5 times, each soaking duration is 16-48 hours, and the soaking temperature is a constant 20-26° C.

Further, step 2) employs sodium hydroxide solution and hydrogen peroxide solution for stepwise detoxification treatment.

Specifically, first soaking in a sodium hydroxide solution, wherein the concentration of the sodium hydroxide solution is 0.5-1.5 mol/L, then soaking in a hydrogen peroxide solution, wherein the concentration of the hydrogen peroxide solution is 30 wt %;

when soaking in the hydrogen peroxide solution, the material-to-liquid weight ratio of intestinal casing to hydrogen peroxide solution is 1:(5-6), the soaking temperature is 20-26° C.; the soaking duration is 16-36 hours, and the water is changed every 45-60 minutes.

Further, in step 5), first soaking the intestinal casing processed in step 4) in a growth factor solution multiple times, then spreading it flat, stacking multiple layers, and pressing to form the membrane.

Preferably, the concentrations of the growth factors in the growth factor solution are respectively:
VEGF: 30-50 μg/g, FGF: 40-70 μg/g, TGF-β1: 15-28 μg/g, BMP: 44-55 μg/g.

When soaking in the growth factor solution, the material-to-liquid weight ratio of intestinal casing to growth factor solution is 1:(3-8), and the number of soaking cycles is 6-8;
in this step, soaking is performed in a constant temperature shaker at a speed of 300-500 rpm and a constant temperature of 30° C.; the soaking duration is 60-90 minutes per cycle, for 6-8 cycles; before each immersion in the growth factor solution, the material needs to be washed with PBS (pH 7.0).

Based on the same inventive concept, the collagen matrix membrane prepared by the above method is applied in the preparation of a periodontal regeneration membrane.

The beneficial effects achieved by adopting the technical solution of the invention are:
the invention uses animal intestinal casing, such as pig intestinal casing, as raw material. After multi-step processing, it prepares a collagen matrix membrane with excellent mechanical properties and a longer degradation cycle, suitable for periodontal treatment. This membrane is conducive to providing sufficient bone regeneration space and time during the initial healing phase of periodontal disease, better meeting clinical application needs.

Moreover, the collagen matrix membrane prepared by the invention is loaded with multiple growth factors beneficial for periodontal tissue growth, thereby facilitating the regeneration and repair of periodontal tissue and improving therapeutic efficacy.

The invention uses low-cost and widely sourced animal intestinal casing as raw material, resulting in low preparation costs.

### BRIEF DESCRIPTION OF THE DRAWINGS

FIG. 1 is a picture of the finished collagen matrix membrane of Embodiment 1 according to the invention.

FIG. 2 is an SEM micrograph of the collagen matrix membrane of Embodiment 1 according to the invention.

FIG. 3 shows pictures of the degradation experiment process for the Embodiment and Comparative Embodiment collagen matrix membranes, where (a) is Comparative Embodiment 1 and (b) is Embodiment 1.

### SPECIFIC EMBODIMENT OF THE INVENTION

In order to make the objectives, technical solutions, and advantages of the invention clearer, the following further describes the invention in detail with reference to embodiments; it should be understood that the specific embodiments described herein are only used to explain the invention and not to limit it.

The technical solution of the invention is a preparation method of a collagen matrix membrane, including the following steps:

1) taking intestinal casing from a pig, cleaning it, and soaking it in water;
in this step, frozen pig intestinal casing can be used. After thawing, cutting into segments approximately 2-5 meters long, cleaning and cutting into long strips. Washing with purified water until no foreign matter is visible externally. Then soaking in water multiple times, each soaking for 2-5 minutes, repeated 2-3 times;

2) soaking the intestinal casing from step 1) in a viral inactivation solution, followed by washing with purified water;
the viral inactivation solution in this step is selected from at least one of sodium hydroxide solution, sodium hypochlorite solution, formic acid, or hydrogen peroxide solution.

Preferably, the viral inactivation solution adopts sodium hydroxide solution and hydrogen peroxide solution. Specifically, sequentially treating with sodium hydroxide solution and hydrogen peroxide solution stepwise. Specifically, first soaking the intestinal casing from step 1) in a sodium hydroxide solution, followed by washing with purified water; then soaking the processed intestinal casing in a hydrogen peroxide solution, followed by washing with purified water;

The alkali solution used in this step is a sodium hydroxide solution with a concentration of 0.5-1.5 mol/L.

When soaking in the alkali solution, the material-to-liquid weight ratio of intestinal casing to alkali solution is 1:(5-6), the soaking temperature is 0-10° C., and the soaking duration is 0.5-2 hours.

This step adopts alkali solution to decompose nucleic acids, dissolve cell membranes and cytoplasmic components, achieving decellularization.

The hydrogen peroxide solution has a concentration of 30 wt %.

In this step, when soaking in the hydrogen peroxide solution, the material-to-liquid weight ratio of intestinal casing to hydrogen peroxide solution is 1:(5-6), the soaking temperature is 20-26° C.; the soaking duration is 16-36 hours, and the water is changed every 45-60 minutes.

The purpose of using hydrogen peroxide in this step is to utilize its strong oxidizing properties to destroy nucleic acid structures, thereby inactivating viruses in animal-derived materials.

3) Degreasing
soaking the intestinal casing processed in step 2) in a degreasing solution multiple times, followed by washing with purified water;
the degreasing solution in step 3) is prepared from chloroform and methanol in a volume ratio of (2-4):1.

The soaking in this step is performed 2-5 times, each soaking duration is 16-48 hours, and the soaking temperature is constant at 20-26° C.

This step adopts chloroform and methanol solution to effectively dissolve and remove fats from the surface of the intestinal casing.

4) Deproteinization

Soaking the intestinal casing processed in step 2) in a sodium dodecyl sulfate solution, followed by washing with purified water;

The sodium dodecyl sulfate solution used in this step has a concentration of 0.1-0.3 wt %.

When soaking in the sodium dodecyl sulfate solution, the material-to-liquid weight ratio of intestinal casing to sodium dodecyl sulfate solution is 1:(5-6), the soaking temperature is constant at 20-26° C., and the soaking duration is 3-9 hours.

This step adopts sodium dodecyl sulfate to dissolve cell membranes, primarily aiming to separate DNA and proteins.

5) Soaking the intestinal casing processed in step 4) in a growth factor solution, then spreading it flat, stacking multiple layers (5-20 layers), pressing to form a membrane, and performing gradient freeze-drying with a freeze dryer. The concentrations of the growth factors in the growth factor solution used in this step are respectively:
VEGF: 30-50 μg/g, FGF: 40-70 μg/g, TGF-β1: 15-28 μg/g, BMP: 44-55 μg/g.

When soaking in the growth factor solution, the material-to-liquid weight ratio of intestinal casing to growth factor solution is 1:(3-8), and the number of soaking cycles is 6-8;
in this step, soaking in the growth factor solution is performed in a constant temperature shaker at a speed of 300-500 rpm and a constant temperature of 30° C.; the soaking duration is 60-90 minutes per cycle, for 6-8 cycles. Before each immersion in the growth factor solution, the material needs to be washed with PBS (pH 7.0).

In this step, a hydraulic cutting press can be used for pressing. Parameters: distance from upper platen to worktable: 5.2 cm, pressing duration: 1.8-2.5 s/time, number of presses: 3-4 times. Using multiple presses in this step, compared to single-press forming, makes the final product less prone to delamination during subsequent use.

The specific operating steps for gradient freeze-drying with a freeze dryer in this step are as follows:
a. Temperature (° C.): −65; Vacuum (Pa): 0; Time (h): 1-4.5;
b. Temperature (° C.): −55; Vacuum (Pa): 0; Time (h): 0.5-1;
c. Temperature (° C.): −30; Vacuum (Pa): 0; Time (h): 0.5-1;
d. Temperature (C): −30; Vacuum (Pa): 26-30; Time (h): 8-12;
e. Temperature (C): −25; Vacuum (Pa): 26-30; Time (h): 8-12;
f. Temperature (C): 0; Vacuum (Pa): 26-30; Time (h): 1-1.5;
g. Temperature (° C.): 5; Vacuum (Pa): 26-30; Time (h): 1-1.5;
h. Temperature (° C.): 5; Vacuum (Pa): 26-30; Time (h): 1-1.5;
i. Temperature (° C.): 16; Vacuum (Pa): 26-30; Time (h): 1-1.5;
j. Temperature (° C.): 16; Vacuum (Pa): 0; Time (h): 1-2.

Further, more specifically, the specific operating steps for gradient freeze-drying are shown in Table 1.

TABLE 1
Gradient Freeze-Drying Operation Steps

| | Temp | Vacuum | |
|---|---|---|---|
| Step | (° C.) | (Pa) | Time (h) |
| 1 | −65 | 0 | 2 |
| 2 | −55 | 0 | 1 |
| 3 | −30 | 0 | 1 |
| 4 | −30 | 28 | 8 |
| 5 | −25 | 28 | 8 |
| 6 | 0 | 28 | 1 |
| 7 | 5 | 28 | 1 |
| 8 | 5 | 28 | 1 |
| 9 | 16 | 28 | 1 |
| 10 | 16 | 0 | 2 |

As shown in Table 1, the gradient freeze-drying operation steps are as follows:
a. Temp (° C.): −65; Vacuum (Pa): 0; Time (h): 2;
b. Temp (C): −55; Vacuum (Pa): 0; Time (h): 1;
c. Temp (° C.): −30; Vacuum (Pa): 0; Time (h): 1;
d. Temp (° C.): −30; Vacuum (Pa): 28; Time (h): 8;
e. Temp (° C.): −25; Vacuum (Pa): 28; Time (h): 8;
f. Temp (° C.): 0; Vacuum (Pa): 28; Time (h): 1;
g. Temp (° C.): 5; Vacuum (Pa): 28; Time (h): 1;
h. Temp (° C.): 5; Vacuum (Pa): 28; Time (h): 1;
i. Temp (° C.): 16; Vacuum (Pa): 28; Time (h): 1;
j. Temp (° C.): 16; Vacuum (Pa): 0; Time (h): 2.

Using the above gradient freeze-drying parameters on the formed membrane sheet, compared to constant temperature freeze-drying, results in a more uniform thickness of the finished product, making it less prone to delamination during subsequent use. Furthermore, gradient freeze-drying has less impact on the internal fiber structure of the product, resulting in stronger water absorption.

Gradient freeze-drying is also more efficient than constant temperature drying, shortening the production cycle.

Relevant performance tests were conducted on the collagen matrix membrane prepared by the invention. The test results are as follows:

The collagen matrix membrane is loaded with multiple growth factors. The content of the loaded growth factors is shown in Table 2 below;

TABLE 2

| Growth Factor Type | Content | |
|---|---|---|
| VEGF | 4000-4500 | (pg/g) |
| FGF | 5900-6600 | (pg/g) |
| TGF-β1 | 3200-3500 | (pg/g) |
| BMP | 9.1-10.0 | (μg/g) |

Thickness of the collagen matrix membrane: 0.1-0.89 mm.

Total protein content of the collagen matrix membrane: 90%-98.5%;

Tensile strength (wet state) of the collagen matrix membrane: 17-35 MPa, tear force (wet state): 5-8 N;

Water absorption of the collagen matrix membrane: 3.5-5.0 g/g;

In vitro degradation time of the collagen matrix membrane: 20-27 days.

The test basis for the above performance is as follows:

YY/T 1794-2021 General Technical Requirements for Oral Collagen Membranes—In vitro degradation test method: using a 0.02 mg/mL collagenase solution to simulate the product use environment. Reference standards for in vitro degradation: GB/T 16886.9-2001 Biological evaluation of medical devices—Part 9: Framework for identification and quantification of potential degradation products. GB/T 16886.13-2001 Biological evaluation of medical devices—Part 13: Identification and quantification of degradation products from polymeric medical devices. YY/T 0474-2004 In vitro degradation test for poly (L-lactide) resins and products for surgical implants.

The usage mode of the invention is: moistening the biological membrane with physiological saline, covering the wound and suture. If delamination occurs during subsequent use, it can affect the healing process.

Embodiment 1: a preparation method of a collagen matrix membrane, including the following steps:

1) taking intestinal casing from a pig, cleaning it, and soaking it in water;
in this step, frozen pig intestinal casing can be used. After thawing, cutting into segments approximately 2-5 meters long, cleaning and cutting into long strips. Washing with purified water until no foreign matter is visible externally. Then soaking in water multiple times, each soaking for 2 minutes, repeated 2 times;

2) soaking the intestinal casing from step 1) in a viral inactivation solution, followed by washing with purified water;
this step sequentially adopts sodium hydroxide solution and hydrogen peroxide solution for stepwise treatment. Specifically, first soaking the intestinal casing from step S1 in a sodium hydroxide solution, followed by washing with purified water; then soaking the processed intestinal casing in a hydrogen peroxide solution, followed by washing with purified water;

The alkali solution used in this step is a sodium hydroxide solution with a concentration of 0.8 mol/L.

When soaking in the alkali solution, the material-to-liquid weight ratio of intestinal casing to alkali solution is 1:6, the soaking temperature is 4° C., and the soaking duration is 90 minutes.

This step adopts alkali solution to decompose nucleic acids, dissolve cell membranes and cytoplasmic components, achieving decellularization.

The hydrogen peroxide solution has a concentration of 30 wt %.

In this step, when soaking in the hydrogen peroxide solution, the material-to-liquid weight ratio of intestinal casing to hydrogen peroxide solution is 1:5, the soaking temperature is 20° C.; the soaking duration is 28 hours, and the water is changed every 60 minutes.

3) soaking the intestinal casing processed in step 2) in a degreasing solution multiple times, followed by washing with purified water;
the degreasing solution in this step is prepared from chloroform and methanol in a volume ratio of 4:1.

The soaking in this step is performed 3 times, with soaking durations of 16 h, 24 h, and 48 h respectively, and the soaking temperature is constant at 20° C.

4) soaking the intestinal casing processed in step 3) in a sodium dodecyl sulfate solution, followed by washing with purified water;
The sodium dodecyl sulfate solution used in this step has a concentration of 0.1 wt %.

When soaking in the sodium dodecyl sulfate solution, the material-to-liquid weight ratio of intestinal casing to sodium dodecyl sulfate solution is 1:5, the soaking temperature is constant at 20° C., and the soaking duration is 8 hours.

5) Soaking the intestinal casing processed in step 4) in a growth factor solution, then spreading it flat, stacking 10 layers, pressing multiple times with a hydraulic cutting press to form a membrane, and performing gradient freeze-drying with a freeze dryer. The concentrations of the growth factors in the growth factor solution used in this step are respectively:
VEGF: 30 μg/g, FGF: 55 μg/g, TGF-β1: 16 μg/g, BMP: 50 μg/g.

When soaking in the growth factor solution, the material-to-liquid weight ratio of intestinal casing to growth factor solution is 1:5, and the number of soaking cycles is 8;
in this step, soaking in the growth factor solution is performed in a constant temperature shaker at a speed of 350 rpm and a constant temperature of 30° C.; the soaking duration is 75 minutes per cycle, for 8 cycles. Before each immersion in the growth factor solution, the material needs to be washed with PBS (pH 7.0).

In this step, a hydraulic cutting press can be used for pressing. Parameters: distance from upper platen to worktable: 5.2 cm, pressing duration: 1.8-1.9 s/time, number of presses: 3 times.

The specific operating steps for gradient freeze-drying with a freeze dryer in this step follow the specific operation in Table 1 above.

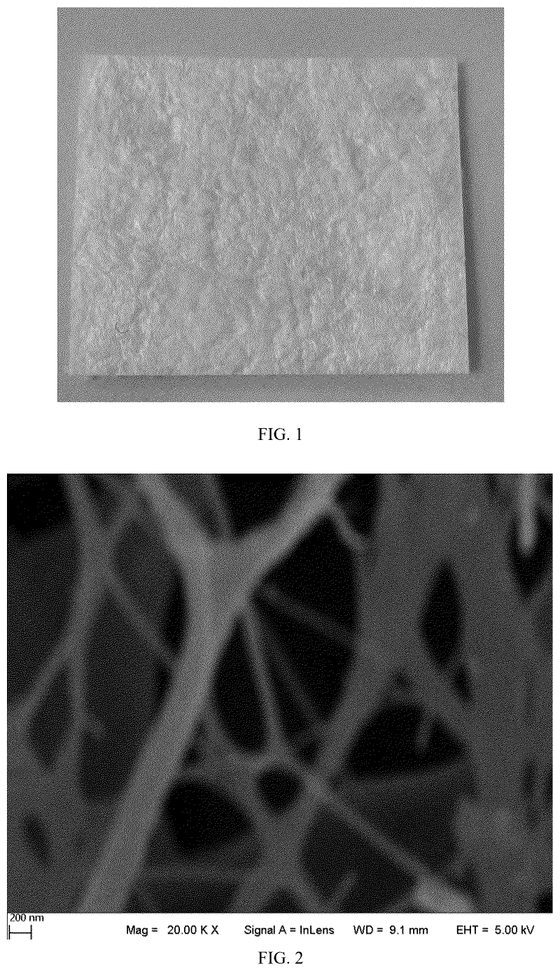

The finished collagen matrix membrane prepared in this embodiment is shown in FIG. 1, and its SEM internal structure is shown in FIG. 2.

Its performance is as follows:
the content of the loaded growth factors is shown in Table 3 below:

TABLE 3

| Growth Factor Type | Content (Average Value) | |
|---|---|---|
| VEGF | 4372.7 | (pg/g) |
| FGF | 6289.8 | (pg/g) |
| TGF-β1 | 3349.4 | (pg/g) |
| BMP | 9.61399 | (μg/g) |

Total protein content of the collagen matrix membrane: 98.5%;
Tensile strength (wet state) of the collagen matrix membrane: 34.75 MPa, tear force (wet state): 7.6 N;
Water absorption of the collagen matrix membrane: 5.0 g/g;
In vitro degradation time of the collagen matrix membrane: 27 days.

Embodiment 2: a preparation method of a collagen matrix membrane, including the following steps:

1) taking intestinal casing from a pig, cleaning it, and soaking it in water;
in this step, frozen pig intestinal casing can be used. After thawing, cutting into segments approximately 2-5 meters long, cleaning and cutting into long strips. Washing with purified water until no foreign matter is visible externally. Then soaking in water multiple times, each soaking for 2 minutes, repeated 2 times;

2) soaking the intestinal casing from step 1) in a viral inactivation solution, followed by washing with purified water;
this step sequentially adopts sodium hydroxide solution and hydrogen peroxide solution for stepwise treatment. Specifically, first soaking the intestinal casing from step S1 in a sodium hydroxide solution, followed by washing with purified water; then soaking the processed intestinal casing in a hydrogen peroxide solution, followed by washing with purified water;
The alkali solution used in this step is a sodium hydroxide solution with a concentration of 0.5 mol/L.

When soaking in the alkali solution, the material-to-liquid weight ratio of intestinal casing to alkali solution is 1:6, the soaking temperature is 4° C., and the soaking duration is 30 minutes.

This step adopts alkali solution to decompose nucleic acids, dissolve cell membranes and cytoplasmic components, achieving decellularization.

The hydrogen peroxide solution has a concentration of 30 wt %.

In this step, when soaking in the hydrogen peroxide solution, the material-to-liquid weight ratio of intestinal casing to hydrogen peroxide solution is 1:5, the soaking temperature is 20° C.; the soaking duration is 28 hours, and the water is changed every 60 minutes.

3) soaking the intestinal casing processed in step 2) in a degreasing solution multiple times, followed by washing with purified water;
the degreasing solution in this step is prepared from chloroform and methanol in a volume ratio of 4:1.

The soaking in this step is performed 3 times, with soaking durations of 16 h, 24 h, and 48 h respectively, and the soaking temperature is constant at 20° C.

4) soaking the intestinal casing processed in step 3) in a sodium dodecyl sulfate solution, followed by washing with purified water;

The sodium dodecyl sulfate solution used in this step has a concentration of 0.1 wt %.

When soaking in the sodium dodecyl sulfate solution, the material-to-liquid weight ratio of intestinal casing to sodium dodecyl sulfate solution is 1:5, the soaking temperature is constant at 20° C., and the soaking duration is 3 hours.

5) Soaking the intestinal casing processed in step 4) in a growth factor solution, then spreading it flat, stacking 10 layers, pressing multiple times with a hydraulic cutting press to form a membrane, and performing gradient freeze-drying with a freeze dryer. The concentrations of the growth factors in the growth factor solution used in this step are respectively:
VEGF: 30 μg/g, FGF: 40 μg/g, TGF-β1: 15 μg/g, BMP: 44 μg/g.

When soaking in the growth factor solution, the material-to-liquid weight ratio of intestinal casing to growth factor solution is 1:5, and the number of soaking cycles is 8;
in this step, soaking in the growth factor solution is performed in a constant temperature shaker at a speed of 350 rpm and a constant temperature of 30° C.; the soaking duration is 75 minutes per cycle, for 8 cycles. Before each immersion in the growth factor solution, the material needs to be washed with PBS (pH 7.0).

In this step, a hydraulic cutting press can be used for pressing. Parameters: distance from upper platen to worktable: 5.2 cm, pressing duration: 1.8-1.9 s/time, number of presses: 3 times.

The specific operating steps for gradient freeze-drying with a freeze dryer in this step follow the specific operation in Table 1 above.

Its performance is as follows:
the content of the loaded growth factors is shown in Table 4 below:

TABLE 4

| Growth Factor Type | Content (Average Value) | |
|---|---|---|
| VEGF | 4058.1 | (pg/g) |
| FGF | 5990.2 | (pg/g) |
| TGF-β1 | 3234.4 | (pg/g) |
| BMP | 9.12085 | (μg/g) |

Total protein content of the collagen matrix membrane: 92.1%;
Tensile strength (wet state) of the collagen matrix membrane: 20.11 MPa, tear force (wet state): 5.9 N;
Water absorption of the collagen matrix membrane: 4.1 g/g;
In vitro degradation time of the collagen matrix membrane: 25 days.

Embodiment 3: a preparation method of a collagen matrix membrane, including the following steps:

1) taking intestinal casing from a pig, cleaning it, and soaking it in water;
in this step, frozen pig intestinal casing can be used. After thawing, cutting into segments approximately 2-5 meters long, cleaning and cutting into long strips. Washing with purified water until no foreign matter is visible externally. Then soaking in water multiple times, each soaking for 2 minutes, repeated 2 times;

2) soaking the intestinal casing from step 1) in a viral inactivation solution, followed by washing with purified water;
this step sequentially adopts sodium hydroxide solution and hydrogen peroxide solution for stepwise treatment. Specifically, first soaking the intestinal casing from step S1 in a sodium hydroxide solution, followed by washing with purified water; then soaking the processed intestinal casing in a hydrogen peroxide solution, followed by washing with purified water;
The alkali solution used in this step is a sodium hydroxide solution with a concentration of 1.5 mol/L.

When soaking in the alkali solution, the material-to-liquid weight ratio of intestinal casing to alkali solution is 1:6, the soaking temperature is 4° C., and the soaking duration is 120 minutes.

This step adopts alkali solution to decompose nucleic acids, dissolve cell membranes and cytoplasmic components, achieving decellularization.

The hydrogen peroxide solution has a concentration of 30 wt %.

In this step, when soaking in the hydrogen peroxide solution, the material-to-liquid weight ratio of intestinal casing to hydrogen peroxide solution is 1:5, the soaking temperature is 20° C.; the soaking duration is 36 hours, and the water is changed every 60 minutes.

3) soaking the intestinal casing processed in step 2) in a degreasing solution multiple times, followed by washing with purified water;
the degreasing solution in this step is prepared from chloroform and methanol in a volume ratio of 4:1.

The soaking in this step is performed 3 times, with soaking durations of 16 h, 24 h, and 48 h respectively, and the soaking temperature is constant at 20° C.

4) soaking the intestinal casing processed in step 3) in a sodium dodecyl sulfate solution, followed by washing with purified water;
The sodium dodecyl sulfate solution used in this step has a concentration of 0.3 wt %.

When soaking in the sodium dodecyl sulfate solution, the material-to-liquid weight ratio of intestinal casing to sodium dodecyl sulfate solution is 1:5, the soaking temperature is constant at 20° C., and the soaking duration is 9 hours.

5) Soaking the intestinal casing processed in step 4) in a growth factor solution, then spreading it flat, stacking 10 layers, pressing multiple times with a hydraulic cutting press to form a membrane, and performing gradient freeze-drying with a freeze dryer. The concentrations of the growth factors in the growth factor solution used in this step are respectively:
VEGF: 50 μg/g, FGF: 70 μg/g, TGF-β1: 28 μg/g, BMP: 55 μg/g.

When soaking in the growth factor solution, the material-to-liquid weight ratio of intestinal casing to growth factor solution is 1:5, and the number of soaking cycles is 8;
in this step, soaking in the growth factor solution is performed in a constant temperature shaker at a speed of 350 rpm and a constant temperature of 30° C.; the soaking duration is 75 minutes per cycle, for 8 cycles. Before each immersion in the growth factor solution, the material needs to be washed with PBS (pH 7.0).

In this step, a hydraulic cutting press can be used for pressing. Parameters: distance from upper platen to worktable: 5.2 cm, pressing duration: 1.8-1.9 s/time, number of presses: 3 times.

The specific operating steps for gradient freeze-drying with a freeze dryer in this step follow the specific operation in Table 1 above.

Its performance is as follows:
the content of the loaded growth factors is shown in Table 5 below:

TABLE 5

| Growth Factor Type | Content (Average Value) | |
|---|---|---|
| VEGF | 4372.7 | (pg/g) |
| FGF | 6289.8 | (pg/g) |
| TGF-β1 | 3349.4 | (pg/g) |
| BMP | 9.61399 | (μg/g) |

Total protein content of the collagen matrix membrane: 94.7%;
Tensile strength (wet state) of the collagen matrix membrane: 29.62 MPa, tear force (wet state): 6.3 N;
Water absorption of the collagen matrix membrane: 4.3 g/g;
In vitro degradation time of the collagen matrix membrane: 21 days.

Comparative Embodiment 1: a preparation method of a collagen matrix membrane, including the following steps:

1) taking intestinal casing from a pig, cleaning it, and soaking it in water;
in this step, frozen pig intestinal casing can be used. After thawing, cutting into segments approximately 2-5 meters long, cleaning and cutting into long strips. Washing with purified water until no foreign matter is visible externally. Then soaking in water multiple times, each soaking for 2 minutes, repeated 2 times;

2) soaking the intestinal casing from step 1) in a viral inactivation solution, followed by washing with purified water;
the viral inactivation solution adopts sodium hydroxide solution and hydrogen peroxide solution. Specifically, sequentially treating with sodium hydroxide solution and hydrogen peroxide solution stepwise. Specifically, first soaking the intestinal casing from step 1) in a sodium hydroxide solution, followed by washing with purified water; then soaking the processed intestinal casing in a hydrogen peroxide solution, followed by washing with purified water;
The alkali solution used in this step is a sodium hydroxide solution with a concentration of 0.8 mol/L.

When soaking in the alkali solution, the material-to-liquid weight ratio of intestinal casing to alkali solution is 1:6, the soaking temperature is 4° C., and the soaking duration is 90 minutes.

This step adopts alkali solution to decompose nucleic acids, dissolve cell membranes and cytoplasmic components, achieving decellularization.

The hydrogen peroxide solution has a concentration of 30 wt %.

In this step, when soaking in the hydrogen peroxide solution, the material-to-liquid weight ratio of intestinal casing to hydrogen peroxide solution is 1:5, the soaking temperature is 20° C.; the soaking duration is 28 hours, and the water is changed every 60 minutes.

3) Degreasing

3) soaking the intestinal casing processed in step 2) in a degreasing solution multiple times, followed by washing with purified water;
the degreasing solution in this step is prepared from chloroform and methanol in a volume ratio of 4:1.

The soaking in this step is performed 3 times, with soaking durations of 16 h, 24 h, and 48 h respectively, and the soaking temperature is constant at 20° C.

4) Deproteinization

4) soaking the intestinal casing processed in step 3) in a sodium dodecyl sulfate solution, followed by washing with purified water;
The sodium dodecyl sulfate solution used in this step has a concentration of 0.1 wt %.

When soaking in the sodium dodecyl sulfate solution, the material-to-liquid weight ratio of intestinal casing to sodium dodecyl sulfate solution is 1:5, the soaking temperature is constant at 20° C., and the soaking duration is 8 hours.

5) Soaking the intestinal casing processed in step 4) in a growth factor solution, then spreading it flat, stacking 10 layers, pressing multiple times with a hydraulic cutting press to form a membrane, and then performing constant temperature freeze-drying.

The concentrations of the growth factors in the growth factor solution used in this step are respectively:
VEGF: 30 μg/g, FGF: 55 μg/g, TGF-β1: 16 μg/g, BMP: 50 μg/g.

When soaking in the growth factor solution, the material-to-liquid weight ratio of intestinal casing to growth factor solution is 1:5, and the number of soaking cycles is 8;
in this step, soaking in the growth factor solution is performed in a constant temperature shaker at a speed of 350 rpm and a constant temperature of 30° C.; the soaking duration is 75 minutes per cycle, for 8 cycles. Before each immersion in the growth factor solution, the material needs to be washed with PBS (pH 7.0).

In this step, a hydraulic cutting press can be used for pressing. Parameters: distance from upper platen to worktable: 5.2 cm, pressing duration: 1.8-1.9 s/time, number of presses: 3 times.

The constant temperature freeze-drying conditions in this Comparative Embodiment are as follows:

Temperature (C): −30° C.; Vacuum (Pa): 28; Time (h): 72.

Its performance is as follows:
the content of the loaded growth factors is shown in Table 6 below:

TABLE 6

| Growth Factor Type | Content (Average Value) | |
|---|---|---|
| VEGF | 3057.3 | (pg/g) |
| FGF | 4610.1 | (pg/g) |
| TGF-β1 | 2450.2 | (pg/g) |
| BMP | 8.13281 | (μg/g) |

Total protein content of the collagen matrix membrane: 86.7%;
Tensile strength (wet state) of the collagen matrix membrane: 9.83 MPa, tear force (wet state): 2.9 N;
Water absorption of the collagen matrix membrane: 2.1 g/g;
In vitro degradation time of the collagen matrix membrane: 16 days.

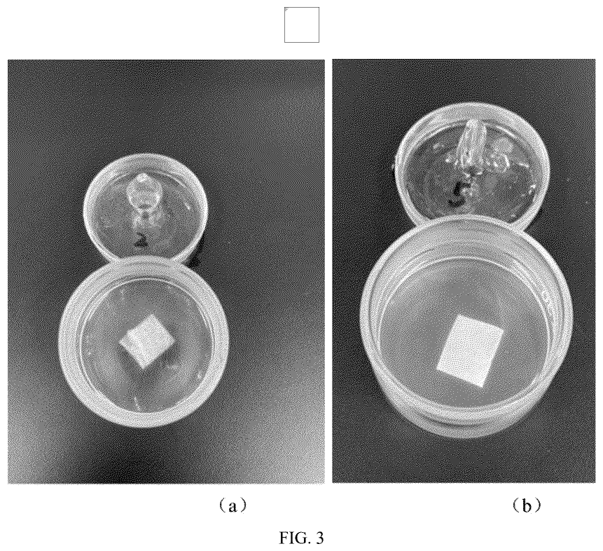

FIG. 3 shows the degradation experiment process for the collagen matrix membranes of Comparative Embodiment 1 and Embodiment 1. As seen in the figure, Comparative Embodiment 1 showed obvious delamination on day 10 of the degradation experiment, while Embodiment 1 did not.

### Comparative Embodiment 2: Existing Product

### Oral Absorbable Biological Membrane (Landu)

Water absorption (average): 3.47 g/g
Tear force (average): 3.94 N
Tensile strength (average): 14.95 MPa
Degradation time: 20 days.

The above descriptions are merely preferred embodiments of the invention. It should be noted that for those of ordinary skill in the art, various improvements and modifications can be made without departing from the principle of the invention, and these improvements and modifications should also be regarded as the protection scope of the invention.

1. A preparation method of a collagen matrix membrane, including the following steps:
1) taking intestinal casing from a mammal, cleaning it, and soaking it in water;
2) soaking the intestinal casing from step 1) in a viral inactivation solution, followed by washing with purified water;
3) soaking the intestinal casing processed in step 2) in a degreasing solution multiple times, followed by washing with purified water;
4) soaking the intestinal casing processed in step 3) in a sodium dodecyl sulfate solution, followed by washing with purified water;
5) spreading the intestinal casing processed in step 4) flat, stacking multiple layers, pressing multiple times to form a membrane, and performing gradient freeze-drying with a freeze dryer.

2. The preparation method of a collagen matrix membrane of claim 1, wherein the mammal in step 1) is selected from one of pig, cow, or sheep.

3. The preparation method of a collagen matrix membrane of claim 1, wherein the viral inactivation solution in step 2) is selected from at least one of sodium hydroxide solution, sodium hypochlorite solution, formic acid, or hydrogen peroxide solution.

4. The preparation method of a collagen matrix membrane of claim 1, wherein the concentration of the sodium dodecyl sulfate solution in step 4) is 0.1-0.3 wt %.

5. The preparation method of a collagen matrix membrane of claim 1, wherein the degreasing solution in step 3) is prepared from chloroform and methanol in a volume ratio of (2-4):1.

6. The preparation method of a collagen matrix membrane of claim 5, wherein step 3) adopts soaking in a degreasing solution 2-5 times, each soaking duration is 16-48 hours, and the soaking temperature is a constant 20-26° C.

7. The preparation method of a collagen matrix membrane of claim 1, wherein step 2) employs sodium hydroxide solution and hydrogen peroxide solution for stepwise detoxification treatment.

8. The preparation method of a collagen matrix membrane of claim 1, wherein in step 5), first soaking the intestinal casing processed in step 4) in a growth factor solution multiple times, then spreading it flat, stacking multiple layers, and pressing to form the membrane.

9. The preparation method of a collagen matrix membrane of claim 8, wherein the concentrations of the growth factors in the growth factor solution are respectively:
VEGF: 30-50 μg/g, FGF: 40-70 μg/g, TGF-β1: 15-28 μg/g, BMP: 44-55 μg/g.

10. An application of the collagen matrix membrane of claim 1 in the preparation of a periodontal regeneration membrane.
